(19) United States
(12) Patent Application Publication
(10) Pub. No.: US 2022/0356441 A1
(43) Pub. Date: Nov. 10, 2022

(54) Reactor for Two-Stage Liquid-Solid State Fermentation of Microorganisms

(71) Applicant: Locus IP Company, LLC, Solon, OH (US)

(72) Inventors: Sean FARMER, Ft. Lauderdale, FL (US); Ken ALIBEK, Solon, OH (US)

(21) Appl. No.: 17/765,547

(22) PCT Filed: Dec. 14, 2020
(86) PCT No.: PCT/US2020/064808

§ 371 Date: Mar. 31, 2022

Related U.S. Application Data
(60) Provisional application No. 62/947,597, filed on Dec. 13, 2019.

(51) Int. Cl. C12N 1/20; C12M 1/00; C12M 1/12; C12M 1/16; C12M 1/36

(52) U.S. Cl. CPC C12N 1/205; C12M 23/58; C12M 23/04; C12M 29/08; C12M 29/18; C12M 21/16; C12M 41/48; C12R 2001/07

(57) ABSTRACT

In preferred embodiments, the subject invention provides two-vessel fermentation systems for producing microbe-based products comprising fungal mycelia and/or spores, and/or bacterial endospores, wherein the systems comprise both a submerged fermentation vessel and a solid state fermentation (SSF) vessel. Advantageously use of the two phases improves the efficiency of producing microorganisms by catering to the different requirements for biomass and/or vegetative cell accumulation as well as the requirements for mycelial growth and/or sporulation.

### CROSS-REFERENCE TO A RELATED APPLICATION

This application claims the benefit of U.S. Provisional Application Ser. No. 62/947,597, filed Dec. 13, 2019, which is incorporated herein by reference in its entirety.

### BACKGROUND OF THE INVENTION

Microorganisms, such as bacteria and fungi, are important for the production of a variety of industrially-relevant chemicals. These microbes and thir by-products are useful in many industries, such as oil production; agriculture; remediation of soils, water and other natural resources; mining; animal feed; waste treatment and disposal; food and beverage preparation and processing; and human health.

One factor limiting the commercialization of microbe-based products has been the cost per propagule density, in which the impracticality of producing microbial products in large scale operations with sufficient inoculum limits the benefits. This is partly due to the difficulties in cultivating microbial products on a large scale.

Two principle forms of microbe cultivation exist for growing bacteria and fungi: liquid submerged fermentation and surface cultivation (solid-state fermentation (SSF)). Both cultivation methods require a medium for the growth of the microorganisms, but the cultivation methods are classified based on the type of substrate used during fermentation (either a liquid or a solid substrate). The growth medium for both types of fermentation typically includes a carbon source, a nitrogen source, salts and other appropriate additional nutrients and trace elements.

Liquid submerged fermentation can be ideally suited for logarithmic growth of microorganisms, enabling a rapid increase in the concentration of the microbes. This method utilizes free-flowing liquid substrates, such as molasses and nutrient broth, into which bioactive compounds can be secreted by the growing microbes. The microorganisms can grow at a logarithmic rate because of the availability of the dissolved nutrients. However, transporting microorganisms produced by submerged cultivation can be complicated and costly, in addition to the difficulty for people to implement the process in the field, e.g., in a remote location where the product will be used.

SSF utilizes solid substrates, such as bran, bagasse, and paper pulp, for culturing microorganisms. The substrates are utilized slowly and steadily, so the same substrate can be used for long fermentation periods. But, because the substrates are utilized more slowly, the cells may not be able to grow at a logarithmic rate. The nutrients can be so limited that the yeast or bacterial cells form spores or endospores, respectively. The formation of spores or endospores is advantageous in a variety of industries. For example, since spores and endospores are resistant to desiccation, the microbes can be transported and stored effectively in this form without the additional complexity and expense of handling microbes in liquid.

Fungi commonly form spores, which are units for reproduction and can be remain viable in adverse growth conditions (e.g., few nutrients or the presence of toxic chemicals). Fungi are often classified based on the differences of the formed spores. For example, in certain fungi, two haploid spores of fungi mate (sexual reproduction) to form a vegetative diploid fungal cell. Other fungi do not mate but instead use spores to establish genetic clones, known as budding. Some undergo budding and mating. The fungal spores may be able to resist various stresses, including pasteurization and desiccation. Fungal sporulation is critical for cell replication; temporal cycles and also environmental factors affect the process.

Certain types of fungal spores can enter dormancy under conditions such as lack of nutrients, low temperature, an unfavorable pH, or the presence of an inhibitor (such as, for example, a plant exudate). The spore will delay germination, and is considered exogenously dormant. Other fungal spores become endogenously dormant, meaning they do not germinate immediately, even under favorable conditions. This can be due to an innate nutrient impermeability or the presence of endogenous inhibitors. Upon a certain environmental or physiological event (e.g., high heat) that allows for nutrients to enter the spore or inhibitors to leach from the spore, dormancy is ended.

In addition to fungi, some bacteria produce endospores, often referred to as spores, yet they are different from eukaryotic spores—endospores are not a means for reproduction. Endospores are often formed under nutrient limitation conditions; however, once nutrients are no longer limited, the bacteria can begin growing again as a vegetative cell. The endospore helps prevent desiccation and the harmful effects caused by UV light, high temperatures, and other stress factors. The ability to withstand temporary nutrient deficiencies or other stress factors is one advantage to using endospore-forming bacteria in various industries.

In some examples, endospore-forming bacteria and spore-forming yeast also produce industrially-efficacious surfactants. Surfactants are chemicals that reduce the surface tension between two substances, often acting as dispersants, emulsifiers, or detergents. Microbially-produced surfactants, referred to as biosurfactants, are of increasing interest in a variety of industries due to their diversity, environmentally-friendly nature, selectivity, and performance in adverse conditions that including high temperature and high salinity. Biosurfactants have excellent surface and interfacial tension reduction properties, as well as other beneficial biochemical properties, which can be useful in applications such as large scale industrial uses.

Biosurfactants can form of micelles, liposomes, or bilayers, providing a physical mechanism to mobilize, for example, oil in a moving aqueous phase. Furthermore, biosurfactants accumulate at interfaces, reducing interfacial tension and leading to the formation of aggregated micellar structures in solution. Advantageously, the ability of biosurfactants to form pores and destabilize biological membranes permits their use as, for example, antimicrobial and hemolytic agents. Thus, there exists an enormous potential for the use of microbes in a variety of industries.

The use of microbe-based products has been greatly limited by difficulties in production, transportation, administration, pricing and efficacy. For example, many microbial agricultural products are applied through irrigation systems; however, the products can clog these systems due to cell size and/or aggregation, and thus require additional processing and grinding of the product into particulates. Additionally, many microbes are difficult to grow and subsequently deploy to agricultural and forestry operations in sufficient quantities to be useful. This problem is exacerbated by losses in viability and/or activity due to processing, formulating, storage, and stabilizing prior to distribution.

Furthermore, once applied, biological products may not thrive for any number of reasons including, for example, insufficient initial cell densities, the inability to compete effectively with the existing microflora at a particular location, and being introduced to soil and/or other environmental conditions in which the microbe cannot flourish or even survive.

Microbe-based compositions could help resolve some of the aforementioned issues faced by the agriculture industry, the oil and gas industry, as well as many others. Thus, there is a need for more efficient cultivation methods for mass production of microorganisms and microbial metabolites.

### BRIEF SUMMARY OF THE INVENTION

This invention relates to the production of microbe-based products for commercial applications. Specifically, the subject invention provides systems and methods for the efficient production of beneficial microbes, as well as for the production of growth by-products of these microbes.

Advantageously, the cultivation methods can be scaled up or down in size. Most notably, the methods can be scaled to an industrial scale, meaning a scale that is capable of supplying microbe-based products in amounts suitable for commercial applications, e.g., oil and/or gas recovery, bioleaching, agriculture, livestock production, and aquaculture. Furthermore, the subject invention can be used as a “green” process for producing microorganisms and their metabolites on a large scale and at low cost, without releasing harmful chemicals into the environment.

In preferred embodiments, the subject invention provides two-stage fermentation systems for producing microbe-based products comprising spore-form bacteria and/or fungi, wherein the systems comprise both a submerged fermentation stage and a solid state fermentation (SSF) stage. Advantageously use of the two stages improves the efficiency of producing microorganisms by catering to the different requirements for biomass and/or vegetative cell accumulation as well as the requirements for sporulation and/or spread of fungal mycelia.

In general, a first vessel is filled with a liquid nutrient medium and then inoculated with a microbial culture. The culture is grown for a period of time to allow for accumulation of microbial biomass and/or vegetative cells in the liquid nutrient medium. In certain embodiments, the vegetative cell concentration reaches about 1×10⁴ to 1×10¹³ cells/m1 in the first vessel. The first vessel is connected to a second vessel designed for SSF. The culture is transferred from the first vessel to the second vessel, which comprises a plurality of smaller chambers therein, each adapted for housing a solid substrate. The microorganisms are grown on a solid substrate in the chambers under conditions that encourage sporulation and/or spreading of fungal mycelia, and then the solid-state culture is harvested from the plurality of chambers and, optionally, dried.

In specific embodiments, the first vessel is a rectangular or cylindrical tank. Preferably, the tank is made of a metal or metal alloy, for example, stainless steel. The tank can have an opening at the top that can be sealed during operation and/or cleaning. Furthermore, the tank can range in volume from a few gallons to thousands of gallons. In some embodiments, the tank can hold about 1 gallon to about 2,000 gallons of liquid.

The first vessel can comprise a mixing system, a temperature control system, water access, an aeration system, and probes for monitoring, e.g., pH, temperature and dissolved oxygen.

In certain embodiments, the first vessel is connected to the second vessel via a plurality of inoculation lines, each of which comprises a tube or a pipe, through which the culture comprising vegetative cells and/or biomass are transferred into the second vessel. Preferably, each of the plurality of inoculation lines leads to and/or is connected to one of the plurality of chambers within the second vessel.

In certain embodiments each of the plurality of chambers is completely separate from each of the others, so as to prevent the spread of contamination between the chambers. For example, in some embodiments, each chamber comprises its own filtered air supply, which can also be used for individualized heating and/or cooling of each chamber. Thus, if one chamber is contaminated, its contents can be removed so that the entire fermentation batch is not contaminated and wasted.

In one embodiment, each chamber is loaded with a solid substrate. An aliquot of the culture is directed through each of the inoculation lines and sprayed onto, or otherwise contacted with, the solid substrate within each of the chambers. In certain embodiments, the system comprises a means for spreading the culture in an even layer over the substrate.

In some embodiments, the chambers within the second vessel are in the form of horizontally-oriented trays with a base and sides, said trays measuring the width and length of the second vessel. The substrate is spread in an even layer over the entire tray. In preferred embodiments, the trays comprise a port that leads to the bottom of the second vessel when opened. The port can be located in a side of the tray or in the base of the tray, and can comprise a removable cover.

The trays are preferably situated in parallel to one another within the second vessel, with ample space between each tray to allow for air flow within each chamber. For example, in some embodiments, the trays can be situated with about 6 inches to about 48 inches of space between one another.

In some embodiments, a rod is rotatably attached to a motor at the top of the second vessel. The rod extends inside the second vessel, from the top of the second vessel to the bottom, passing through an opening in the center of each of the trays, and rotates when the motor is running.

In certain embodiments, within each chamber of the second vessel, the portion of the rod therein comprises a spreading mechanism comprising a flat face and an edge, such as a squeegee or a blade made of metal, rubber, silicone or plastic. The spreading mechanism extends outward from the rod towards the perimeter of the tray and is situated so that its flat face is perpendicular, or near-perpendicular, to the tray.

As the rod rotates, the spreading mechanism rotates. The height of the spreading mechanism above the base of the tray can be adjusted depending upon what stage of fermentation is occurring. As an exemplary embodiment, the spreading mechanism can be used to spread about 1 to 6 inches of solid substrate over a tray; thus, the height of the spreading mechanism is adjusted to about 1 to 6 inches above the base of the tray.

As another exemplary embodiment, the spreading mechanism is used to spread the inoculant culture over the solid substrate layer; thus, the height of the spreading mechanism is adjusted to, for example, about 0.25 to about 2 inches above the height of the solid substrate layer.

As another exemplary embodiment, the spreading mechanism is used as a scraping mechanism, wherein the tray's port is opened by removing its cover, and the height of the spreading mechanism is lowered continuously while rotating so that the substrate containing the culture is scraped from the tray and directed through the port.

In certain alternative embodiments, the chambers of the second vessel are in the form of hollow cylinders comprised of, for example, screen or mesh, preferably oriented in parallel with one another within the vessel. In some embodiments, the screen or mesh is further surrounded by a solid cylinder, made of, for example, metal or plastic, which can further comprise removable covers at one or both ends. The substrate is pre-spread onto the screen or mesh with space inside so as to retain a hollow chamber, and then the chamber is loaded into the second vessel. In certain embodiments, the cylindrical chambers are situated in a circle, ellipse or triangle, or square, with about 0.5 inches to about 12 inches of space between each chamber.

In some embodiments, the second vessel comprises a revolving solid cylinder having cylindrical openings in which the cylindrical chambers are loaded. In some embodiments, as the revolving cylinder rotates, each chamber passes by a blade or plug mechanism, which is inserted into the chamber in order to either spread inoculant over the substrate, or scrape the substrate and/or mature culture out of the chamber and into the bottom of the second vessel.

In certain embodiments, the second vessel comprises a collection vessel at the bottom, into which the culture and, optionally, substrate from each of the plurality of chambers is collected after maturation of the spores and/or mycelia.

In preferred embodiments, the subject invention provides methods for cultivating microorganisms using the subject systems. The microorganisms are grown using submerged fermentation in the first vessel until the biomass content and/or vegetative cell concentration reaches a certain point. Then, the culture is spread onto the solid substrate in each chamber of the second vessel, and is subjected to conditions that encourage spore formation and/or fungal mycelial growth. Once the culture matures sufficiently within each chamber, the culture and, optionally, substrate are collected into a collection vessel at the bottom of the second vessel, harvested therefrom, and optionally, processed further.

In certain embodiments, the microbe-based products produced according to these methods can be used for agriculture, for example, as soil amendments, biopesticides, and/or biofertilizers.

Advantageously, the subject systems and methods reduce the time and materials required for large-scale production of microbial biomass and spore-form microorganisms.

### BRIEF DESCRIPTION OF THE FIGURES

FIGS. 1A-1B show a second vessel according to an embodiment of the subject invention. 1A shows an outer view of the second vessel and 1B show a deconstructed view of the second vessel, wherein the plurality of chambers are in the form of horizontally-oriented trays.

FIGS. 2A-2B show a second vessel according to an embodiment of the subject invention wherein the plurality of chambers are in the form of hollow cylinders oriented in parallel to one another on a rotating rod or carousel (2B). 2A shows the interior of one of the chambers comprising a cylindrical screen upon which microbial culture is grown.

### DETAILED DESCRIPTION

This invention relates to the production of microbe-based products for commercial applications. Specifically, the subject invention provides systems and methods for the efficient production of beneficial microbes, as well as for the production of growth by-products of these microbes.

In preferred embodiments, the subject invention provides two-stage fermentation systems for producing microbe-based products comprising spore-form bacteria and/or fungi, wherein the systems comprise both a submerged fermentation stage and a solid state fermentation (SSP) stage. Advantageously use of the two stages improves the efficiency of producing microorganisms by catering to the different requirements for biomass and/or vegetative cell accumulation as well as the requirements for sporulation and/or mycelial spreading.

### Selected Definitions

As used herein, a “biofilm” is a complex aggregate of microorganisms, such as bacteria, wherein the cells adhere to each other and/or to a surface using an extracellular polysaccharide matrix. The cells in biofilms are physiologically distinct from planktonic cells of the same organism, which are single cells that can float or swim in liquid medium.

As used herein, “co-cultivation” means cultivation of more than one strain or species of microorganism in a single fermentation system. In some instances, the microorganisms interact with one another, either antagonistically or symbiotically, resulting in a desired effect, e.g., a desired amount of cell biomass growth or a desired amount of metabolite production. In one embodiment, this antagonistic or symbiotic relationship can result in an enhanced effect, for example, the desired effect can be magnified when compared to what results from cultivating only one of the chosen microorganisms on its own. In an exemplary embodiment, one microorganism causes and/or stimulates the production of one or more metabolites by another microorganism, e.g., a Myxococcus sp. stimulates a Bacillus sp. to produce a biosurfactant.

As used herein, “enhancing” refers to improving and/or increasing.

As used herein, “fermentation” refers to cultivation or growth of cells under controlled conditions. The growth could be aerobic or anaerobic.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain), and in some embodiments, in association with a carrier.

In certain embodiments, purified compounds are at least 60% by weight the compound of interest. Preferably, the preparation is at least 75%, more preferably at least 85%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state or in spore form, or a mixture of both. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells or spores may be totally absent, or present at, for example, a concentration of at least 1×10⁴, 1×10⁵, 1×10⁶, 1×10⁷, 1×10⁸, 1×10⁹ 1×10¹⁰, 1×10¹¹ or 1×10¹² or more CFU per milliliter of the composition.

The subject invention further provides “microbe-based products,” which are products that are to be applied in practice to achieve a desired result. The microbe-based product can be simply the microbe-based composition harvested from the microbe co-cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “reduces” means a negative alteration of at least 1%, 5%, 10%, 25%, 50%, 75%, or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and/or dispersants. A “biosurfactant” is a surface-active substance produced by a living cell.

The transitional teiin “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited components(s).

Unless specifically stated or obvious from context, as used herein, the term “or” is understood to be inclusive. Unless specifically stated or obvious from context, as used herein, the terms “a,” “and,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Fermentation System Design

In preferred embodiments, the subject invention provides two-stage fermentation systems for producing microbe-based products comprising spore-form bacteria and/or fungi, wherein the systems comprise both a submerged fermentation stage and a solid state fermentation (SSF) stage. Advantageously use of the two stages improves the efficiency of producing microorganisms by catering to the different requirements for biomass and/or vegetative cell accumulation as well as the requirements for sporulation and/or fungal mycelial spreading.

In general, a first vessel is filled with a liquid nutrient medium and inoculated with a microbial culture. The culture is grown for a period of time to allow for accumulation of microbial biomass and/or vegetative cells. In certain embodiments, the vegetative cell concentration reaches about 1×10⁴ to 1×10¹³ cells/ml in the first vessel. The first vessel is connected to a second vessel designed for SSF. The culture is transferred from the first vessel to the second vessel, which comprises a plurality of smaller chambers therein, each of which is adapted to house a solid substrate. The microorganisms are grown on the solid substrate under conditions that encourage sporulation and/or spreading of fungal mycelia, and then the solid-state culture is harvested from the plurality of chambers and, optionally, dried.

In specific embodiments, the first vessel is a rectangular or cylindrical tank. Preferably, the tank is made of a metal or metal alloy, for example, stainless steel. The tank can have an opening at the top that can be sealed during operation and/or cleaning. Furthermore, the tank can range in volume from a few gallons to thousands of gallons. In some embodiments, the tank can hold about 1 gallon to about 2,000 gallons of liquid.

The first vessel can comprise a mixing system, a temperature control system, water access, an aeration system, and probes for monitoring, e.g., pH, temperature and dissolved oxygen. In one embodiment, the first vessel is a fermentation reactor according to the description in international publication WO 2019/133555A1, which is incorporated herein by reference in its entirety.

In preferred embodiments, the first vessel utilizes a chaotic mixing scheme to circulate the culture and ensure highly efficient mass exchange. The chaotic mixing scheme uses an internal mixing apparatus as well as an external circulation system.

In one embodiment, the internal mixing apparatus comprises a mixing motor located at the top of the tank. The motor is rotatably attached to a metal shaft that extends into the tank and is fixed with an impeller to help propel tank liquid from the top of the tank to the bottom of the tank and to ensure efficient mixing and gas dispersion throughout the culture. In one embodiment, the metal shaft with the impeller rotates on a diagonal axis (e.g., an axis at 15 to 60° from vertical).

In one embodiment, the impeller is a standard four-blade Rushton impeller. In one embodiment, the impeller comprises an axial flow aeration turbine and/or a small marine propeller. In one embodiment, the impeller design comprises customized blade shapes to produce increased turbulence.

In one embodiment, the chaotic mixing scheme further utilizes an external circulation system. In preferred embodiments, the external circulation system doubles as a temperature control system. Advantageously, in certain embodiments, the external circulation system obviates the need for a double-walled tank or an external temperature control jacket.

In one embodiment, the external circulation system comprises two highly efficient external loops comprising inline heat exchangers. In one embodiment, the heat exchangers are shell-and-tube heat exchangers. Each loop is fitted with its own circulation pump.

The two pumps transport liquid from the bottom of the tank at, for example, 250 to 400 gallons per minute, through the heat exchangers, and hack into the top of the tank. Advantageously, the high velocity at which the culture is pumped through the loops helps prevent cells from caking on the inner surfaces thereof.

The loops can be attached to a water source and, optionally, a chiller, whereby the water is pumped with a flow rate of about 10 to 15 gallons per minute around the culture passing inside the heat exchangers, thus increasing or decreasing temperature as desired. In one embodiment, the water controls the temperature of the culture without ever contacting the culture.

The first vessel can further comprise an aeration system capable of providing filtered air to the culture. The aeration system can, optionally, have an air filter for preventing contamination of the culture. The aeration system can function to keep the air level over the culture, the dissolved oxygen (DO), and the pressure inside the tank, at desired (e.g., constant) levels.

In certain embodiments, the first vessel can be equipped with a unique sparging system, through which the aeration system supplies air. Preferably, the sparging system comprises stainless steel injectors that produce microbubbles. In an exemplary embodiment, the spargers can comprise from 4 to 10 aerators, comprising stainless steel microporous pipes (e.g., having tens or hundreds of holes 1 micron or less in size), which are connected to an air supply. The unique microporous design allows for proper dispersal of oxygen throughout the culture, while preventing contaminating microbes from entering the culture through the air supply.

In some embodiments, the first vessel is controlled by a programmable logic controller (PLC). In certain embodiments, the PLC has a touch screen and/or an automated interface. The PLC can be used to start and stop the reactor system, and to monitor and adjust, for example, temperature, DO, and pH, throughout biomass accumulation.

The first vessel can be equipped with probes for monitoring fermentation parameters, such as, e.g., pH, temperature and DO levels. The probes can be connected to a computer system, e.g., the PLC, which can automatically adjust fermentation parameters based on readings from the probes.

In certain embodiments, the DO is adjusted continuously as the microorganisms of the culture consume oxygen and reproduce. For example, the oxygen input can be increased steadily as the microorganisms grow, in order to keep the DO constant at about 30% (of saturation).

In certain embodiments, the first vessel is connected to the second vessel via a plurality of inoculation lines, each of which comprises a tube or a pipe, through which the culture comprising vegetative cells and/or biomass are transferred into the second vessel.

The second vessel preferably comprises a plurality of smaller chambers, each of which is adapted for housing a solid substrate. Preferably, each of the plurality of inoculation lines leads to and/or is connected to one of the chambers within the second vessel.

In certain embodiments each of the plurality of chambers is completely separate from each of the others, so as to prevent the spread of contamination between the chambers. For example, in some embodiments, each chamber comprises its own filtered air supply, which can also be used for individualized heating and/or cooling of each chamber. Thus, if one chamber is contaminated, its contents can be removed so that the entire fermentation batch is not contaminated and wasted.

In one embodiment, a solid substrate is spread into each chamber. An aliquot of the culture, in liquid form, is directed through each of the inoculation lines and sprayed onto, or otherwise contacted with, the solid substrate within each of the chambers. In certain embodiments, the system comprises a means for spreading the culture in an even layer over the substrate.

In preferred embodiments, the substrate according to the subject methods serves as a three-dimensional scaffold structure comprising a plurality of internal and external surfaces on which microbes can grow and, ultimately, form spores and/or mycelia.

In certain embodiments, the substrate is comprised of a plurality of individual solid items, e.g., pieces, morsels, grains, or particles. The individual solid items are arranged so as to create the scaffold structure (or matrix). Preferably, the solid items are capable of substantially retaining their shape and/or structure, even in the presence of a liquid. In some embodiments, the matrix is capable of substantially retaining its shape and/or structure as a whole, even though the solid substrate therein may be mixed with a liquid.

In some embodiments, substantially retaining shape and/or structure means retaining shape and/or structure to such a degree that the internal and external surfaces of the matrix, or total surface area thereof, are not compromised and remain exposed for microbes to colonize, and, in preferred embodiments, exposed to air and/or other gases.

In one embodiment, the plurality of solid items are preferably solid pieces, morsels, grains, or particles of foodstuff. The foodstuff can include one or more of, for example, rice, legumes, corn and other grains, oats and oatmeal, pasta, wheat bran, flours or meals (e.g., corn flour, nixtamilized corn flour, partially hydrolyzed corn meal), and/or other similar foodstuff to provide surface area for the microbial culture to grow and/or feed on.

In one embodiment, the foodstuff is a legume. Legumes include beans, nuts, peas and lentils. Examples of legumes according to the subject invention include but are not limited to chickpeas, runner beans, fava beans, adzuki beans, soybeans, Anasazi beans, kidney beans, butter beans, haricots, cannellini beans, flageolet beans, pinto beans, borlotti beans, black beans, peanuts, soy nuts, carob nuts, green peas, snow peas, snap peas, split peas, garden peas, and black, red, yellow, orange, brown and green lentils.

In one embodiment, wherein the matrix comprises grains of rice, the matrix substrate can be prepared by mixing rice grains and a liquid medium comprising additional salts and/or nutrients to support microbial growth.

In some embodiments, the rice can be, for example, long grain, medium grain, short grain, white (polished), brown, black, basmati, jasmine, wild, arborio, matta, rosematta, red cargo, sticky, sushi, Valencia rice, and any variation or combination thereof.

In certain embodiments, the type of foodstuff utilized as the solid substrate will depend upon which microbe is being cultivated. For example, in one embodiment, Trichoderma spp. can be cultivated efficiently using corn flour or modified forms thereof, and in another embodiment, Bacillus spp. can be cultivated efficiently using rice. These microbial taxa are not limited to these specific substrates, however.

In certain embodiments, the substrate is mixed with water prior to being spread into the chambers. In certain embodiments, the substrate is mixed with a liquid nutrient medium comprising, for example, maltose or another carbon source, yeast extract or another source of protein, and sources of minerals, potassium, sodium, phosphorous and/or magnesium. Alternatively, in some embodiments, no additional nutrients are added to the solid substrate.

In some embodiments, the foodstuff in the matrix can also serve as a source of nutrients for the microbes. Furthermore, the matrix can provide increased access to oxygen supply when a microorganism requires cultivation under aerobic conditions.

In one embodiment, when a motile microorganism is being cultivated, the method can further comprise applying a motility enhancer, such as potato extract and/or banana peel extract, to the matrix to increase the speed of microbial motility and distribution throughout the matrix. Sporulation enhancers can also be added to the substrate to increase the speed of sporulation.

Sterilization of the chambers and substrate can be performed after the substrate has been spread and prior to inoculation with the liquid culture. Sterilization can be performed by autoclave or any other means known in the art.

In some embodiments, each of the chambers of the second vessel can comprise an aeration system to provide slow motion air supply and/or temperature control within in each chamber. In some embodiments, individual chambers can comprise their own aeration systems. For example, in one embodiment, one chamber comprises an inlet and an outlet, wherein an air pump supplies air into the chamber through tubing attached to the inlet, and then the air exits the chamber through tubing attached to the outlet.

In some embodiments, the chambers within the second vessel are in the form of horizontally-oriented, trays with a base and sides, said trays measuring the width and length of the second vessel.

FIGS. IA-1B. The substrate is spread in an even layer over the entire tray. In preferred embodiments, the trays comprise a port that leads to the bottom of the second vessel when the port is opened. The port can be located in a side of the tray or in the base of the tray, and can comprise a removable cover.

The trays are preferably situated in parallel to one another within the second vessel, with ample space between each tray to allow for air flow within each chamber. For example, in some embodiments, the trays can be situated with about 6 inches to about 48 inches of space between one another.

In some embodiments, a rod is rotatably attached to a motor at the top of the second vessel. The rod extends inside the second vessel from the top of the second vessel to the bottom, passing through an opening in the center of each of the trays, and rotates when the motor is running.

In certain embodiments, within each chamber of the second vessel, the portion of the rod therein comprises a spreading mechanism comprising a flat face and an edge, such as a squeegee or a blade made of metal, rubber, silicone or plastic. The spreading mechanism extends outward from the rod towards the perimeter of the tray and is situated so that its flat face is at a 90° to 45° angle to the tray.

As the rod rotates, the spreading mechanism rotates. The height of the spreading mechanism above the base of the tray can be adjusted depending upon what stage of fermentation is occurring. As an exemplary embodiment, the spreading mechanism can be used to spread about 1 to 6 inches of solid substrate over a tray; thus, the height of the spreading mechanism is adjusted to about 1 to 6 inches above the base of the tray.

As another exemplary embodiment, the spreading mechanism is used to spread the inoculant culture over the solid substrate layer; thus, the height of the spreading mechanism is adjusted to, e.g., about 0.25 to about 2 inches above the height of the solid substrate layer.

As another exemplary embodiment, the spreading mechanism is used as a scraping mechanism, wherein the tray's port is opened by removing its cover, and the height of the spreading mechanism is lowered in order to scrape the culture from the substrate and direct it through the port. In some embodiments, the spreading mechanism is lowered continuously while rotating so that the substrate containing the culture is gradually scraped from the tray and directed through the port.

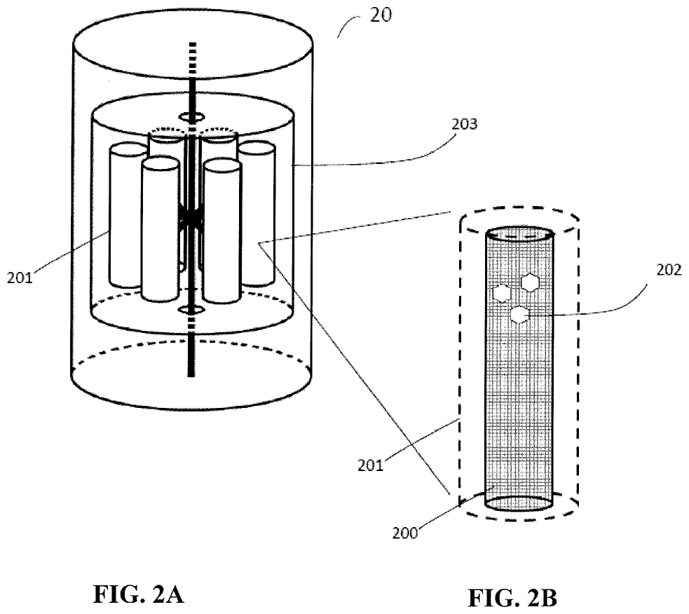

In certain alternative embodiments, the chambers of the second vessel are in the form of hollow cylinders comprised of, for example, screen or mesh, preferably oriented in parallel with one another within the vessel. FIG. 2. In some embodiments, the screen or mesh is further surrounded by a solid cylinder, made of, for example, metal or plastic, which can further comprise removable covers at one or both ends. The substrate is pre-spread onto the screen or mesh with space inside so as to retain a hollow chamber, and then the chamber is loaded into the second vessel. In certain embodiments, the cylindrical chambers are situated in a circle, ellipse or triangle, or square, with about 0.5 inches to about 12 inches of space between each chamber.

In some embodiments, the second vessel comprises a revolving solid cylinder having cylindrical openings in which the cylindrical chambers are loaded. In some embodiments, as the revolving cylinder rotates, each chamber passes by a blade or plug mechanism, which is inserted into the chamber in order to either spread inoculant over the substrate, or scrape the substrate and/or mature culture out of the chamber and into the bottom of the second vessel.

In certain embodiments, the second vessel comprises a collection vessel at the bottom, into which the culture and, optionally, substrate from each of the plurality of chambers is collected after maturation of the culture.

### Methods and Operation of the System

In one embodiment, the subject invention provides materials and methods for the production of biomass (e.g., viable cellular material, vegetative cells), spore-form microorganisms, fungal mycelia, as well as growth by-products of these microorganisms.

In preferred embodiments, the methods utilize the two-vessel systems of the subject invention. The microorganisms are grown using submerged fermentation in the first vessel until the biomass content and/or vegetative cell concentration reaches a certain point. Then, the culture is spread onto the solid substrate in each chamber of the second vessel, and is subjected to conditions that encourage spore formation and/or fungal mycelial growth. Once the culture matures sufficiently within each chamber, the culture and, optionally, substrate are collected into a collection vessel at the bottom of the second vessel, harvested therefrom, and optionally, processed further.

Advantageously, the subject systems and methods reduce the time and materials required for large-scale production of microbial biomass, spore-form microorganisms, and fungal filaments and/or mycelia.

In certain embodiments, the microbe-based products produced according to these methods can be used for agriculture, for example, as soil amendments, biopesticides, and/or biofertilizers.

In preferred embodiments, the method of cultivating a microorganism and/or producing a microbial growth by-product comprises two stages. In certain embodiments, stage (1) comprises filling the first vessel of the subject systems with a liquid nutrient medium; inoculating the liquid nutrient medium with a microorganism; and cultivating the microorganism to accumulate a desired amount of cell biomass and/or vegetative cells.

In one embodiment, the liquid nutrient medium comprises a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

In one embodiment, the liquid nutrient medium comprises a carbon source. The carbon source can be a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sunflower oil, sesame oil, and/or linseed oil; etc. These carbon sources may be used independently or in a combination of two or more.

In one embodiment, growth factors and trace nutrients for microorganisms are included in the liquid nutrient medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included in the liquid nutrient medium. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In preferred embodiments, the microorganism is a bacterium or a fungus. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a fungus, which includes yeasts. Yeast and fungal species according to the current invention, include Aureobasidium (e.g., A. pullulans), Blakeslea, Candida (e.g., C. apicola, C. bombicola, C. nodaensis), Cryptococcus, Debaryontyces (e.g., D. hansenii), Entomophthora, Hanseniaspora, (e.g., H. uvarum), Hansenula, Issatchenkia, Kluyveromyces (e.g., K. phaffii), Lentinula edodes, Mortierella, Mycorrhiza, Meyerozyma (M. aphidis, M. guilliermondii), Penicillium, Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. reesei, T. guizhouse, T. harzianum, T, hamatum, T. viride), Ustilago (e.g., U. maydis), Wickerhamontyces (e.g., W. anomalus), Williopsis (e.g., W. mrakii), Zygosaccharomyces (e.g., Z. hailii), and others.

In exemplary embodiments, the fungus is Wickerhamontyces anomalus, Starmerella bombicola, Saccharomyces boulardii, Pseudozyma aphidis and/or a Pichiayeast (e.g., Pichia occidentalis, Pichia kudriavzevii and/or Pichia guilliermondii (Meyerozyma guilliermondii)).

In another exemplary embodiment, the microorganism is Lentinula edodes, Pleurotus ostreatus, or a Trichoderma spp. fungus (e.g., T. harzianum, guizhouse, T. viride, T, hamatum, and/or T. reesei).

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firMUS, B. laterosporus, B. licheniformis, B. megaterium, B. mucilaginosus, B. polymyxa, B. subtilis), Frateuria (e.g., F. aurantia), Microbacterium (e.g., M. laevaniformans), myxobacteria (e.g., Myxococcus xanthus, Stignalella aurcintiaca, Sorangium cellulosum, Minicystis rosea), Paenibacillus polymyxa, Pantoea (e.g., P. agglomerans), Pseudomonas (e.g., P. aeruginosa, P. chlororaphis, P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In one embodiment, the microorganism is bacteria, such as Pseudomonas chlororaphis, Azotobacter vinelandii, or a Bacillus spp. bacterium, such as, for example, B. subtilis and/or B. amyloliquefaciens (e.g., B. amyloliquefaciens NRRL B-67928).

In a specific embodiment, the Bacillus is B. amyloliquefaciens strain NRRL B-67928 (“B. amy”). A culture of the B. amyloliquefaciens “B. amy” microbe has been deposited with the Agricultural Research Service Northern Regional Research Laboratory (NRRL), 1400 Independence Ave., S.W., Washington, D.C., 20250, USA. The deposit has been assigned accession number NRRL B-67928 by the depository and was deposited on Feb. 26, 2020.

The subject culture has been deposited under conditions that assure that access to the culture will be available during the pendency of this patent application to one determined by the Commissioner of Patents and Trademarks to be entitled thereto under 37 CFR 1.14 and 35 U.S.C. 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In one embodiment, the microorganism is a myxobacterium, or slime-forming bacteria.

Specifically, in one embodiment, the myxobacterium is a Myxococcus spp. bacterium, e.g., M. xanthus.

In one embodiment, two or more microorganisms are co-cultivated using the subject system in order to produce enhanced amounts of metabolites, such as biosurfactants. For example, M. xanthus and B. amyloliquefaciens can be co-cultivated in order to produced enhanced amounts of lipopeptide biosurfactants.

In some embodiments, stage (1) of the method of cultivation can comprise adding acids and/or antimicrobials in the liquid nutrient medium before, and/or during the cultivation process to protect the culture against contamination. These can include, for example, antibiotics (e.g., streptomycin, ampicillin, tetracycline) and/or biosurfactants (e.g., glycolipids, lipopeptides).

stage (1) of the method can comprise providing oxygenation to the growing culture in the first vessel. The oxygenated air may be filtered ambient air supplied through mechanisms including air spargers for supplying bubbles of gas to liquid for dissolution of oxygen into the liquid, and impellers for mechanical agitation of liquid and air bubbles.

The pH of the liquid nutrient medium in the first vessel should be suitable for the microorganism of interest. Buffers, and pH regulators, such as carbonates and phosphates, may be used to stabilize pH of the liquid nutrient medium near a preferred value. When metal ions are present in high concentrations, use of a chelating agent in the medium may be necessary.

The microbes can be grown in the first vessel in planktonic form or as biofilm. In the case of biofilm, the vessel may have within it a substrate (e.g., corn flour) upon which the microbes can be grown in a biofilm state. The system may also have, for example, the capacity to apply stimuli (such as shear stress) that encourages and/or improves the biofilm growth characteristics.

In one embodiment, stage (1) of the method is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In preferred embodiments, stage (1)_of the method comprises operating the first vessel for an amount of time to achieve a desired biomass content and/or vegetative cell concentration in the liquid nutrient medium. The biomass content of the liquid nutrient medium may be, for example, from 5 g/l to 180 g/l or more, or from 10 g/l to 150 g/l. The cell concentration may be, for example, at least 1×10⁴ to 1×10¹³, 1×10⁵to 1×10¹², 1×10⁶to 1×10⁷, or 1×10⁷to 1×10¹⁰cells/ml.

In exemplary embodiments, stage (1) occurs for about 24 hours to 7 days, or about 36 hours to about 5 days.

In preferred embodiments, upon reaching a desired biomass content and/or vegetative cell concentration during stage (1), the method comprises carrying out stage (2).

Stage (2) of the subject invention generally comprises transferring a portion of the culture produced during stage (1) into the second vessel of the two-vessel system and continuing to cultivate the microorganism using solid-state fermentation until the microorganism sporulates.

More specifically, in preferred embodiments, stage (2) of the subject methods comprises loading the chambers of the second vessel with solid substrate (e.g., corn flour and/or rice mixed with water) and inoculating the substrate in each chamber with an aliquot of the culture produced during stage (1). In certain embodiments, a pump directs an aliquot of the culture through the inoculation lines that connect the first vessel and the chambers within the second vessel, and contacts the aliquot of culture with the solid substrate.

Inoculation can be achieved by spraying or pipetting, where the end of the inoculation lines that are connected to the second vessel's chambers comprise a dropper, a spray valve, or a pipette. In certain embodiments, the aliquots are equal to one another in volume. An aliquot can be, for example, about 1 mL to about 5 L of liquid culture.

Stage (2) of the method can further comprise incubating the culture for an amount of time to allow the culture to grow through the substrate and/or to form spores. In preferred embodiments, spore-form microorganisms reach or approach 90-100% sporulation.

In some embodiments, when the culture comprises bacteria, the fermentation conditions are tailored such that endospore formation is encouraged. Typically, a bacterium will form endospores under nutritive stress. Thus, in certain embodiments, the solid substrate is not supplemented with any additional nutrient medium, thereby “starving” the bacteria of carbon and nitrogen sources and encouraging sporulation.

In some embodiments, when the culture comprises fungi, the conditions are tailored such that mycelial growth is encouraged. Use of SSF is especially advantageous for mycelial growth, given that in nature, filamentous fungi grow on the ground, decomposing vegetation under naturally ventilated conditions. Therefore, SSF enables the mycelium to spread on the surface of solid compounds through which air can flow. Additionally, the substrate may be sprayed regularly throughout fermentation (e.g., once a day, once every other day, once per week) with sterilized liquid nutrient medium to increase fungal growth. Furthermore, by utilizing a solid substrate that forms an air-permeable matrix, and/or by circulating air throughout the chambers and substrate, fungal growth is encouraged, including production of reproductive spores.

In some embodiments, when production of dormant fungal spores is desired, the conditions can be tailored to encourage dormancy. For example, water and nutrient supply can be reduced, pH can be adjusted to unfavorable levels, germination inhibitors can be included in the substrate, and/or air supply can be limited.

The temperature within the second vessel is preferably kept between about 15-60° C. The exact temperature range will vary depending upon the microorganism and/or form thereof that is being produced. In preferred embodiments, the amount of incubation time in phase 2 is from 1 day to 14 days, more preferably, from 2 days to 10 days.

After phase 2 is complete, the solid-state culture can be harvested from the second vessel. In certain embodiments, the entire substrate with the culture can be removed from the chambers and collected in the collection vessel at the bottom of the second vessel. In other embodiments, just the culture is harvested from the substrate and collected in the collection vessel.

In certain embodiments, the collected culture, and optionally substrate, are removed from the collection vessel and blended together to produce a microbial slurry. In one embodiment, the microbial slurry is homogenized and dried to produce a dry microbe-based product. Drying can be performed using standard methods in the art, including, for example, spray drying, lyophilization, or freeze drying. In one embodiment, the dried product has approximately 3% to 6% moisture retention.

In one embodiment, the microbial slurry can be utilized directly, without drying or processing. In another embodiment, the microbial slurry can be mixed with water to form a liquid microbe-based product.

In some embodiments, the various formulations of microbe-based product produced according to the subject methods can be stored prior to their use.

In one embodiment, the systems and methods of the subject invention can be used to produce a microbial metabolite, wherein the microbial slurry is mixed with water or another solvent, and this slurry-solvent mixture is then filtered to separate solid portions of the mixture from liquid portions. The extracted liquid, which comprises the microbial metabolite, can then be purified further, if desired, using, for example, centrifugation, rotary evaporation, microfiltration, ultrafiltration and/or chromatography. The metabolite content produced by the method can be, for example, at least 20%, 30%, 40%, 50%, 60%, 70%, 80%, 90% or 95% by weight.

The metabolites and/or growth by-products can be, for example, biosurfactants, enzymes, proteins, ethanol, lactic acid, beta-glucan, peptides, metabolic intermediates, polyunsaturated fatty acid, and lipids. Specifically, in one embodiment, the method can be used to produce a biosurfactant.

In one embodiment, phase 1 of the subject methods is carried out continuously or quasi-continuously, and phase 2 is carried out as a batch process. In this embodiment, a portion of the culture produced in the first vessel is removed at a certain time and transferred to the chambers of the second vessel. Biomass with viable cells remains in the first vessel, and can be supplemented with additional nutrients and/or inoculant as needed. Phase 2 is begun once inoculation occurs from the first vessel, and upon reaching a desired cell or spore count within the second vessel, the entire batch is harvested and collected. New substrate can then be added to the chambers of the second vessel, and then the process begins again.

In an alternative embodiment, both phase 1 and phase 2 are continuous or quasi-continuous, after the initial transfer of culture from vessel 1 to vessel 2. In this embodiment, a portion of the culture produced in the first vessel is removed at a certain time and transferred to the chambers of the second vessel. Biomass with viable cells remains in the first vessel, and can be supplemented with additional nutrients and/or inoculant as needed. Phase 2 is begun once the initial inoculation occurs from the first vessel, and upon reaching a desired cell or spore count within any one of the chambers of the second vessel, that culture of that chamber can be harvested and collected. New substrate can then be added to the chamber that was harvested, and an aliquot of culture from the first vessel is used to inoculate the new substrate. Thus, the method can be carried out indefinitely as individual chambers are inoculated, cultivated, harvested and replaced.

### Preparation of Microbe-Based Products

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

In one embodiment, the different strains are not mixed together, but are applied to a plant and/or its environment as separate microbe-based products.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

The pH of the microbe-based composition should be suitable for the microorganism(s) of interest. In a preferred embodiment, the pH of the composition is about 3.5 to 7.0, about 4.0 to 6.5, or about 5.0.

In one embodiment, additional components such as an aqueous preparation of a salt, such as sodium bicarbonate or carbonate, sodium sulfate, sodium phosphate, sodium biphosphate, can be included in the formulation.

In certain embodiments, an adherent substance can be added to the composition to prolong the adherence of the product to plant parts. Polymers, such as charged polymers, or polysaccharide-based substances can be used, for example, xanthan gum, guar gum, levan, xylinan, gellan gum, curdlan, pullulan, dextran and others.

In preferred embodiments, commercial grade xanthan gum is used as the adherent. The concentration of the gum should be selected based on the content of the gum in the commercial product. If the xanthan gum is highly pure, then 0.001% (w/v—xanthan gum/solution) is sufficient.

In one embodiment, glucose, glycerol and/or glycerin can be added to the microbe-based product to serve as, for example, an osmoticum during storage and transport. In one embodiment, molasses can be included.

In one embodiment, prebiotics can be added to and/or applied concurrently with the microbe-based product to enhance microbial growth. Suitable prebiotics, include, for example, kelp extract, fulvic acid, chitin, humate and/or humic acid. In a specific embodiment, the amount of prebiotics applied is about 0.1 L/acre to about 0.5 L/acre, or about 0.2 L/acre to about 0.4 L/acre.

In one embodiment, specific nutrients are added to and/or applied concurrently with the microbe-based product to enhance microbial inoculation and growth. These can include, for example, soluble potash (K2O), magnesium, sulfur, boron, iron, manganese, and/or zinc. The nutrients can be derived from, for example, potassium hydroxide, magnesium sulfate, boric acid, ferrous sulfate, manganese sulfate, and/or zinc sulfate.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a citrus grove). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a citrus grove), for example, within 300 miles, 200 miles, or even within 100 miles. Advantageously, this allows for the compositions to be tailored for use at a specified location. The formula and potency of microbe-based compositions can be customized for specific local conditions at the time of application, such as, for example, which soil type, plant and/or crop is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve GHG management.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention.

### Example 1

### Second Vessel Design

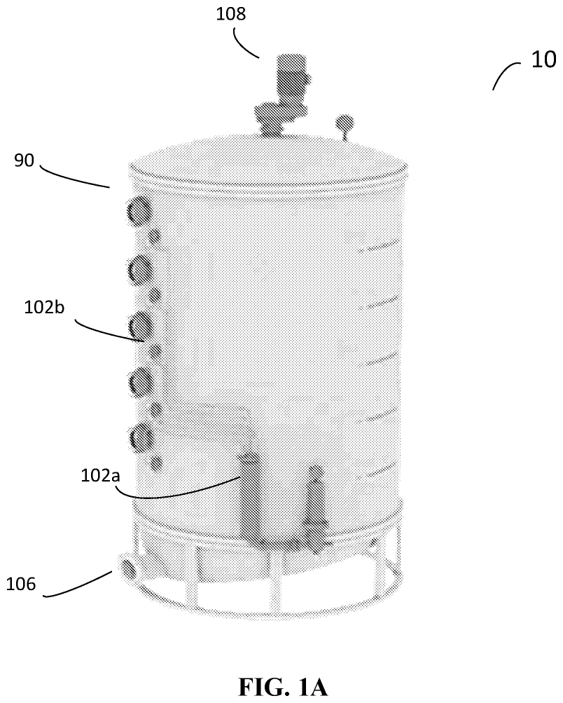

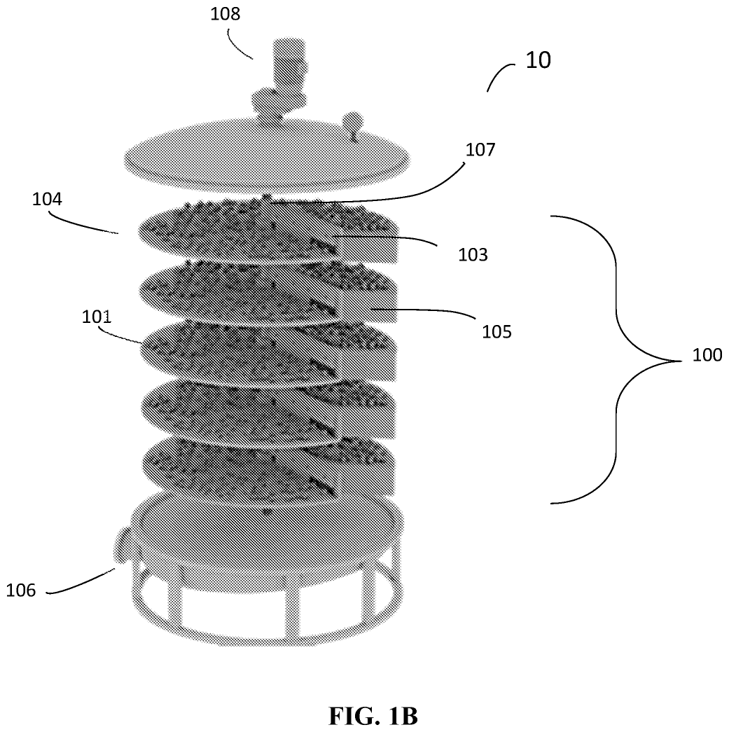

Referring to FIGS. 1A-1B, the second vessel 10 according to the subject invention preferably comprises a plurality of smaller chambers 100, each of which is adapted for housing a solid substrate 101.

In certain embodiments each of the plurality of chambers 100 is completely separate from each of the others, so as to prevent the spread of contamination between the chambers 100. In one embodiment, a solid substrate is spread 101 into each chamber 100. An aliquot of the culture, in liquid form, is directed through each of the inoculation lines 90 and sprayed onto, or otherwise contacted with, the solid substrate 101 within each of the chambers 100. In certain embodiments, the system comprises a means for spreading the culture in an even layer over the substrate 103.

In some embodiments, the second vessel 10 can comprise an aeration system 102a to provide slow motion air supply and/or temperature control within in each chamber. In some embodiments, each individual chamber can comprise its own air supply 102b.

In some embodiments, the chambers 100 within the second vessel 10 are in the form of horizontally-oriented, trays 104, said trays measuring approximately the width and length of the second vessel 10. The substrate 101 is spread in an even layer over the entire tray 104. In preferred embodiments, the trays 104 comprise a port 105 that leads to the bottom of the second vessel 10 when the port is opened. At the bottom of the second vessel 10 is a collection vessel 106.

The trays 104 are preferably situated in parallel to one another within the second vessel 10, with ample space between each tray 104 to allow for air flow within each chamber 100. For example, in some embodiments, the trays 104 can be situated with about 6 inches to about 48 inches of space between one another.

In some embodiments, a rod 107 is rotatably attached to a motor 108 at the top of the second vessel 10. The rod 107 extends inside the second vessel 10 from the top of the second vessel 10 to the bottom, passing through an opening in the center of each of the trays 104, and rotates when the motor 108 is running.

In certain embodiments, within each chamber 100 of the second vessel 10, the portion of the rod 106 therein comprises a spreading mechanism 103 comprising a flat face and an edge, such as a squeegee or a blade made of metal, rubber, silicone or plastic. The spreading mechanism 103 extends outward from the rod 107 towards the perimeter of the tray 104 and is situated so that its flat face is at a 90° to 45° angle to the tray 104.

As the rod 107 rotates, the spreading mechanism 103 rotates. The height of the spreading mechanism 103 above the base of the tray can be adjusted depending upon what stage of fermentation is occurring.

Referring to FIGS. 2A-2B, in certain alternative embodiments, the chambers of the second vessel 20 are in the form of hollow cylinders 200 comprised of, for example, screen or mesh, preferably oriented in parallel with one another within the vessel 20. In some embodiments, the screen or mesh is further surrounded by a solid cylinder 201, made of, for example, metal or plastic, which can further comprise removable covers at one or both ends. The substrate 202 is pre-spread onto the screen or mesh cylinder 200 with space inside so as to retain a hollow chamber, and then the cylinder 200 is loaded into the second vessel 20.

In some embodiments, the second vessel comprises a revolving solid cylinder 203 having cylindrical openings in which the cylindrical chambers 200 are loaded. In some embodiments, as the revolving cylinder 203 rotates, each chamber 202 passes by a blade or plug mechanism (not pictured), which is inserted into the chamber 200 in order to either spread inoculant over the substrate 202, or scrape the substrate 202 and/or mature culture out of the chamber 200 and into the bottom of the second vessel 20.

1. A system for producing microorganisms, the system comprising a first vessel and a second vessel,
said first vessel comprising a tank, a mixing system, a sparging system, and a programmable logic controller (PLC) to monitor and adjust fermentation parameters, wherein said tank is adapted to be filled with a liquid nutrient medium, and wherein the mixing system comprises an internal mixing apparatus and an external circulation system, said external circulation system also functioning as a temperature control system; and
said second vessel comprising a plurality of chambers adapted to house a solid substrate, said chambers each comprising a closeable port, and a collection vessel at a bottom portion of the second vessel to which the closeable ports lead,
wherein the first vessel is connected to the second vessel by a plurality of inoculation lines comprising tubing or piping.

2. The system of claim 1, wherein each of the plurality of inoculation lines is connected directly to one of the plurality of chambers.

3. The system of claim 1, wherein the external circulation system comprises a first and a second external loop, each comprising a shell and tube heat exchanger,
wherein the first and second loop are each attached to a water source and a chiller and fitted with a pump that transports liquid from the bottom of the tank, through the heat exchangers, and back into the top of the tank.

4. The system of claim 1, wherein the sparging system comprises multiple stainless steel microporous aerators,
wherein the microporous aerators each comprise a stainless steel pipe comprising a plurality of holes 1 micron in diameter or less, said pipe attached to an air supply pipe.

5. The system of claim 1, wherein the PLC is connected to a pH probe, a dissolved oxygen probe and a temperature probe and is programmed to automatically implement adjustments to pH, DO and temperature.

6. The system of claim 1, wherein the plurality of chambers in the second vessel are horizontally-oriented trays.

7. The system of claim 6, wherein the second vessel comprises a rod rotatably attached to a motor, said rod extending from the top of the second vessel to the bottom of the second vessel, through each of the chambers, and said rod comprising a spreading means within each chamber that rotates while the rod rotates and spreads and/or scrapes the contents of the chambers.

8. The system of claim 1, wherein the plurality of chambers in the second vessel are hollow cylinders oriented in parallel to one another.

9. The system of claim 1, wherein each of the plurality of chambers comprises an air supply.

10. A method for producing a microorganism using the system of any of claim 1, the method comprising:
filling the first vessel with a liquid nutrient medium;
inoculating the liquid nutrient medium with a microorganism to produce a liquid culture;
cultivating the liquid culture to reach a desired cell biomass and/or vegetative cell concentration;
spreading a solid substrate into a chamber of the second vessel;
directing an aliquot of the liquid culture out of the first vessel, through the inoculation line, and into the chamber, producing a solid-state culture;
cultivating the solid-state culture for an amount of time and under conditions that encourage growth and/or sporulation of the microorganism;
directing the solid-state culture through the chamber's closable port and into the collection vessel;
harvesting the culture from the collection vessel; and
optionally, processing the solid-state culture.

11. The method of claim 10, wherein the microorganism is selected from Trichoderma harzianum, Trichoderma guizhouse, Wickerhamomyces anomalus, Pseudomonas chlororaphis, Saccharomyces boulardii, Debaryomyces hansenii, Meyerozyma guilliermondii, Bacillus amyloliquefaciens, Bacillus licheniformis, Bacillus subtilis, Myxococcus xanthus, Azotobacter vinelandii and Frateuria aurantia.

12. The method of claim 11, wherein the microorganism is B. amyloliquefaciensNRRL B-67928.
